(19) United States
(12) Patent Application Publication
(10) Pub. No.: US 2024/0099992 A1
(43) Pub. Date: Mar. 28, 2024

(54) COMPOSITIONS AND METHODS FOR TREATING CANCER

(71) Applicant: Yanping KONG, Merrimack, NH (US)

(72) Inventors: Yanping Kong, Merrimack, NH (US); Jinhong Liu, Merrimack, NH (US)

(21) Appl. No.: 18/272,939

(22) PCT Filed: Jan. 28, 2022
(86) PCT No.: PCT/US22/14379

§ 371 Date: Jul. 18, 2023

Related U.S. Application Data
(60) Provisional application No. 63/144,690, filed on Feb. 2, 2021.

(51) Int. Cl. A61K 31/132; A61K 45/06; A61P 35/00

(52) U.S. Cl. CPC A61K 31/132; A61K 45/06; A61P 35/00

(57) ABSTRACT

The invention provides pharmaceutical compositions of methylenediamine and methods thereof for treating various types of cancer (e.g., lung cancer, liver cancer, skin cancer, ovarian cancer, prostate cancer, breast cancer and blood cancer).

### PRIORITY CLAIMS AND RELATED APPLICATIONS

This application claims the benefit of priority to U.S. Provisional Application No. 63/144,690, filed Feb. 2, 2021, the entire content of which is incorporated herein by reference for all purposes.

### TECHNICAL FIELD OF THE INVENTION

The invention generally relates to novel therapeutic methods and pharmaceutical compositions for treating cancer. More particularly, the invention relates to pharmaceutical compositions of methylenediamine and uses thereof for treating various types of cancer (e.g., lung cancer, liver cancer, skin cancer, ovarian cancer, prostate cancer, breast cancer and blood cancer).

### BACKGROUND OF THE INVENTION

Cancer is a group of diseases involving abnormal cell growth that can invade or spread to other parts of the body. As of 2019, about 18 million new cases occur annually and caused about 8.8 million deaths. The most common types of cancer in males are lung cancer, prostate cancer, colorectal cancer, and stomach cancer. In females, the most common types are breast cancer, colorectal cancer, lung cancer, and cervical cancer. While many treatment options for cancer exist, including surgery, chemotherapy, radiation therapy, hormonal therapy, targeted therapy and palliative care, cancer remains a top health threat.

Cancer cells contain gene mutations and exhibit rapid growth as well as abnormal metabolism. Processing of nutrients in cancer cells is significantly different from that in normal cells or correspondent benign tumors. Cancer cells have elevated levels of glucose uptake and can utilize pre-formed, diet-derived fatty acids from the bloodstream to accelerate their growth. The evolving of endocytosis in cancer cells may be related to gene profiling change. Our previous studies demonstrated that liver cancer and breast cancer cells, unlike normal cells can take up large nucleic acids from their microenvironment likely via endocytosis. (Kong, et al. 2017 Biological Research 50(2):1-7.) Cancer cells require rapid uptake of nutrients to match their high proliferation rate. Some cancers, such as breast cancer, liver cancer and lung cancer, exhibit more aggressive growth (high proliferation) compared to other types such as thyroid cancer that grow slowly. The rapidly growing cancers require their cells to have high rate of uptake of nutrients.

The therapeutics and methods currently available for treating cancer are inadequate. There remains an urgent and ongoing need for novel and improved therapeutics to effectively treat cancers and related diseases and conditions.

### SUMMARY OF THE INVENTION

The invention is based in part on the unexpected discovery of therapeutic methods and pharmaceutical compositions, as demonstrated herein, that can be used to treat a range of cancers, such as lung cancer, liver cancer, skin cancer, ovarian cancer, prostate cancer, breast cancer and blood cancer. In particular, disclosed herein is a method using methylenediamine (e.g., methylenediamine dihydrochloride) in treatment of cancer and/or prevention or delay recurrence of cancer. Methylenediamine may be used alone or in combination with a variety of other agents.

In one aspect, the invention generally relates to a method for treating cancer, or a related disease or condition. The method comprises administering to a subject in need thereof a therapeutically effective amount of a pharmaceutical composition comprising methylenediamine, or a pharmaceutically acceptable form thereof.

In another aspect, the invention generally relates to a method for reducing the risk of or delaying recurrence of cancer after remission or surgery. The method comprises administering to a subject in need thereof a therapeutically effective amount of a pharmaceutical composition comprising methylenediamine, or a pharmaceutically acceptable form thereof.

In yet another aspect, the invention generally relates to a method for preventing recurrence of cancer after remission or surgery. The method comprises administering to a subject in need thereof a therapeutically effective amount of a pharmaceutical composition comprising methylenediamine, or a pharmaceutically acceptable form thereof.

In yet another aspect, the invention generally relates to a method for suppressing growth of cancer cells. The method comprises administering to a subject in need thereof a pharmaceutical composition comprising methylenediamine, or a pharmaceutically acceptable form thereof.

In yet another aspect, the invention generally relates to a pharmaceutical composition comprising methylenediamine, or a pharmaceutically acceptable form thereof, in a therapeutically effective amount and a pharmaceutically acceptable excipient, carrier, or diluent.

In yet another aspect, the invention generally relates to a unit dosage form comprising a pharmaceutical composition disclosed herein.

In yet another aspect, the invention generally relates to a kit comprising a unit dosage form of the invention and a unit form of a second therapeutic agent and instructions for administration thereof.

In yet another aspect, the invention generally relates to use of methylenediamine, or a pharmaceutically acceptable form thereof, for the manufacture of a medicament for the treatment of cancer (e.g., a solid tumor), or a related disease or condition.

In yet another aspect, the invention generally relates to use of methylenediamine, or a pharmaceutically acceptable form thereof, for treating cancer (e.g., a solid tumor), or a related disease or condition.

### BRIEF DESCRIPTION OF THE DRAWINGS

FIG. 1 shows exemplary data of methylenediamine dihydrochloride (Md in the figure) in suppression of dextran's uptake by H1299 cells (left, lung cancer) and Snu499 cells (right, liver cancer). The red color is dextran in the cancer cells. After adding methylenediamine dihydrochloride at 0.5 mM concentration, it significantly suppressed the uptake of dextran by the cancer cells (much less red color in the cells with methylenediamine dihydrochloride compared with the cells without methylenediamine dihydrochloride).

FIG. 2 shows exemplary data from a proliferation study using MTT assay (Sigma). From left to right are different cancer cell lines. C8161 and MUM2C are melanoma; SKOV3 and OVACA3 are ovarian cancer. ScNAP and DU145 are prostate cancer. H1299 and A549 are lung cancer. MDA231 is breast cancer. SNU499 is liver cancer. For each cell line, 7 columns with different color represent different concentration of methylenediamine dihydrochloride. From left to right: 0 μM, 1 μM, 50 μM, 100 μM, 250 μM, 500 μM and 1 mM. With the dose of methylenediamine dihydrochloride from low to high, the cells were suppressed with a dose dependent pattern, i.e., the higher the dose the more the suppression (the shorter column represents more suppression).

FIG. 3 shows exemplary data from a study of apoptosis induced by methylenediamine dihydrochloride. With methylenediamine dihydrochloride at 500 μM concentration, it significantly shifted the fluorescent peak to the right which means more cells have fluorescent (more fluorescent means more cells in apoptosis). Left: melanoma; right: lung cancer; top: control; middle: low dose (100 μM); bottom: high dose (500 μM) of methylenediamine dihydrochloride. The data showed that methylenediamine dihydrochloride induced apoptosis of cancer cells in a dose dependent manner.

FIG. 4 shows exemplary data on significant suppression of 3 blood cancer cell lines (Jurkat, HL-60 and MV-411) by methylenediamine hydrochloride. The four columns represent number of living cells at different concentration of methylenediamine hydrochloride, from left to right: 0, 50 μM, 100 μM and 500 μM. When concentration increased, fewer living cells were observed.

### DEFINITIONS

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

When trade names are used herein, the trade name includes the product formulation, the generic drug, and the active pharmaceutical ingredient(s) of the trade name product, unless otherwise indicated by context.

Ranges provided herein are understood to be shorthand for all of the values within the range. For example, a range of 1 to 14 is understood to include any number, combination of numbers, or sub-range from the group consisting 1, 2, 3, 4, 5, 6, 7, 8, 9, 10, 11, 12, 13 or 14.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

As used herein, the terms “disease”, “disorder” or “condition” are used interchangeably herein to refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Prodrugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “prevent”, “preventing”, or “prevention” refer to a method for precluding, delaying, averting, or stopping the onset, incidence, severity, or recurrence of a disease or condition. For example, a method is considered to be a prevention if there is a reduction or delay in onset, incidence, severity, or recurrence of a disease or condition or one or more symptoms thereof in a subject susceptible to the disease or condition as compared to a subject not receiving the method. The disclosed method is also considered to be a prevention if there is a reduction or delay in onset, incidence, severity, or recurrence of one or more symptoms of a disease or condition in a subject susceptible to the disease or condition after receiving the method as compared to the subject's progression prior to receiving treatment. The reduction or delay in onset, incidence, severity, or recurrence of cancer can be about a 5, 10, 20, 30, 40, 50, 60, 70, 80, 90, 100%, or any amount of reduction in between.

Prevention and the like do not mean preventing a subject from ever getting the specific disease or disorder. Prevention may require the administration of multiple doses. Prevention can include the prevention of a recurrence of a disease in a subject for whom all disease symptoms were eliminated, or prevention of recurrence in a relapsing-remitting disease.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the term “therapeutically effective amount” refers to the dose of a therapeutic agent or agents sufficient to achieve the intended therapeutic effect with minimal or no undesirable side effects. A therapeutically effective amount can be determined by a skilled physician, e.g., by first administering a low dose of the pharmacological agent(s) and then incrementally increasing the dose until the desired therapeutic effect is achieved with minimal or no undesirable side effects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

Any compositions or methods disclosed herein can be combined with one or more of any of the other compositions and methods provided herein.

Isotopically-labeled compounds are also within the scope of the present disclosure. As used herein, an “isotopically-labeled compound” refers to a presently disclosed compound including pharmaceutical salts thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alignates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### DETAILED DESCRIPTION OF THE INVENTION

The invention provides a novel approach to treatment of various types of cancer. The therapeutic methods and pharmaceutical compositions disclosed herein can benefit cancer patients in terms of reduced risk of recurrence after remission or surgery, increased survival rate and improved treatment outcome. Methods and compositions of the invention can be used to treat a range of cancers, such as lung cancer, liver cancer, skin cancer, ovarian cancer, prostate cancer and breast cancer.

The uptake of nutrients is crucial for rapid growth of cancer cells. Without wishing to be bound by the theory, large nutrient molecules from outside cells can form non-covalent bonds with the polar molecules on the cancer cell's surface to initiate endocytosis. Methylenediamine is able to interfere such bonding and thus suppress the process of uptake.

As disclosed herein, methylenediamine dihydrochloride was tested for suppression of nutrient uptake by cancer cells and for its impact on proliferation of cancer cells. Without wishing to be bound by the theory, another mechanism of action is that methylenediamine can significantly induce apoptosis of cancer cells which is an important way to treat cancer, which may be secondary to or independent of the suppression of cancer cells' uptake. In addition, in vivo study was performed to test the compound against a cancer model. The results confirmed that methylenediamine dihydrochloride was effective in suppression of nutrient uptake by cancer cells and inhibited the proliferation of cancer cells. Furthermore, ethylenediamine dihydrochloride was effective in treating cancers in a mouse cancer model.

Methylenediamine (diaminomethane) has the following chemical formula:

NH₂—CH₂—NH₂

Methylenediamine may be employed in any pharmaceutically acceptable form, for example, in the form of an acid addition salt. Exemplary acid addition salts include the dihydrochloride salt or methylenediamine dihydrochloride (CH₂(NH₂)₂·2HCl).

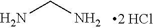

In one aspect, the invention generally relates to a method for treating cancer, or a related disease or condition. The method comprises administering to a subject in need thereof a therapeutically effective amount of a pharmaceutical composition comprising methylenediamine, or a pharmaceutically acceptable form thereof.

In another aspect, the invention generally relates to a method for reducing the risk of or delaying recurrence of cancer after remission or surgery. The method comprises administering to a subject in need thereof a therapeutically effective amount of a pharmaceutical composition comprising methylenediamine, or a pharmaceutically acceptable form thereof.

In yet another aspect, the invention generally relates to a method for preventing recurrence of cancer after remission or surgery. The method comprises administering to a subject in need thereof a therapeutically effective amount of a pharmaceutical composition comprising methylenediamine, or a pharmaceutically acceptable form thereof.

In yet another aspect, the invention generally relates to a method for suppressing growth of cancer cells. The method comprises administering to a subject in need thereof a pharmaceutical composition comprising methylenediamine, or a pharmaceutically acceptable form thereof.

In certain embodiments, methylenediamine is in the form as a pharmaceutically acceptable salt.

In certain embodiments, methylenediamine is in the form of an acid addition salt of methylenediamine (e.g., methylenediamine dihydrochloride).

In certain embodiments, the cancer that is treated is a solid tumor, for example, selected from the group consisting of: lung cancer, liver cancer, skin cancer, ovarian cancer, prostate cancer and breast cancer.

In certain embodiments, the cancer that is treated is a blood cancer, for example, selected from the group consisting of: leukemia, lymphoma, and myeloma.

In certain embodiments, the route of administration is oral, subcutaneous, intramuscular, intratumoral, intravenous, or inhaled administration. In certain embodiments, the route of administration is oral.

In certain embodiments, methylenediamine dihydrochloride is administered at a daily dosage in the range of about 10 mg to about 1,000 mg (e.g., about 10 mg to about 500 mg, about 10 mg to about 100 mg, about 50 mg to about 1,000 mg, about 100 mg to about 1,000 mg, about 500 mg to about 1,000 mg) for a time period of about 7 to about 180 days (e.g., about 7 to about 60 days, about 7 to about 90 days, about 7 to about 120 days). In certain embodiments, the daily dosage is fixed. In certain embodiments, the daily dosage is adjusted based on the subject's response to treatment.

In certain embodiments, methylenediamine dihydrochloride is administered at an enhancing (rising) daily dosage, i.e., increasing daily doses (e.g., from about 10 mg to about 1,000 mg) over a period of time (e.g., about 7 to about 180 days), each adjusted based on the subject's response to treatment. In certain embodiments, rising daily dosage involves doubling the daily dose every other day. In certain embodiments, rising daily dosage involves doubling the daily dose every three days. In certain embodiments, rising daily dosage involves doubling the daily dose every week.

In certain embodiments, a method of the invention further comprises administering to the subject a second therapy or a second therapeutic agent.

In certain embodiments, administration of methylenediamine is given in combination with one or more of chemotherapy, hormone therapy, radiation therapy, and immunotherapy. In certain embodiments, the subject is administered a chemotherapeutic agent. In certain embodiments, the subject is administered a hormonal therapeutic agent. In certain embodiments, the subject is administered radiation therapy. In certain embodiments, the subject is administered immunotherapy.

In certain embodiments, the second therapeutic agent is a small molecule agent.

In certain embodiments, the second therapeutic agent is a protein or antibody.

In certain embodiments, the second therapeutic agent is cell therapy.

In yet another aspect, the invention generally relates to a pharmaceutical composition comprising methylenediamine, or a pharmaceutically acceptable form thereof, in a therapeutically effective amount and a pharmaceutically acceptable excipient, carrier, or diluent.

In certain embodiments, methylenediamine is in the form as a pharmaceutically acceptable salt.

In certain embodiments, methylenediamine is in the form of an acid addition salt (e.g., methylenediamine dihydrochloride).

In yet another aspect, the invention generally relates to a unit dosage form comprising a pharmaceutical composition disclosed herein.

In certain embodiments, the unit dosage form is suitable for oral administration.

In certain embodiments, the unit dosage form is in the form of a tablet or capsule.

In certain embodiments, the unit dosage form is in the form of a liquid solution or suspension.

In yet another aspect, the invention generally relates to a kit comprising a unit dosage form of the invention and a unit form of a second therapeutic agent and instructions for administration thereof.

In certain embodiments, the second therapeutic agent is an agent for chemotherapy. In certain embodiments, the second therapeutic agent is an agent for hormone therapy. In certain embodiments, the second therapeutic agent is an agent for immunotherapy.

In yet another aspect, the invention generally relates to use of methylenediamine, or a pharmaceutically acceptable form thereof, for the manufacture of a medicament for the treatment of cancer (e.g., a solid tumor), or a related disease or condition.

In yet another aspect, the invention generally relates to use of methylenediamine, or a pharmaceutically acceptable form thereof, for treating cancer (e.g., a solid tumor), or a related disease or condition.

In certain embodiments, methylenediamine is used in combination with a second therapy or a second therapeutic agent (e.g., chemotherapy, hormone therapy, radiation therapy, or immunotherapy agents).

Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-F (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylmelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (Angew Chem. Intl. Ed. Engl. (1994) 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esonibicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (doxetaxel; Rhone-Poulene Rorer, Antony, France); chlorambucil; GEMZAR® (gemcitabine); 6-thioguanine; mercaptopurine; methotrexate; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylomithine (DMFO); retinoids such as retinoic acid; and pharmaceutically acceptable salts, acids and derivatives of any of the above.

The following examples are meant to be illustrative of the practice of the invention, and not limiting in any way.

### EXAMPLES

Methylenediamine (dihydrochloride salt) was tested in 2 cancer cell lines for inhibition of uptake by cancer cells (Example 1). This drug was tested for suppression of cancer cells proliferation (Example 2). Methylenediamine dihydrochloride also induced apoptosis (cell death) in melanoma and lung cancer (Example 3). Suppression of blood cancer was observed in 3 cell lines (Example 4). In addition, an efficacy study was performed using an animal model for liver cancer (Example 5). The dose in the animal treatment was calculated to forecast relevant doses for human.

### Example 1. Methylenediamine Dihydrochloride Inhibited Uptake of Fluorescent Dextran by Cancer Cells

Regents: Fluorescent Dextran, Alexa Fluor 600; Clathrin heavy chain antibody as catalog number of AF 488 (MA1065A488); Hoechst #62249 and Blocker buffer in PBS were all from Thermo Fisher. Dextran was used as a marker to test the uptake of the cancer cells.

Cancer cell lines of H1299 (lung cancer) and SNU499 (liver cancer) were cultured with DMEM+10% FBS in Mat Tek 35 mm dish with 14 mm bottom microwell at 5×10⁴/plate for 24 hours and then washed with DMEM. Fluorescent Dextran with or without methylenediamine hydrochloride at 100 μM in DMEM was added, 30 minutes at 37° C. in CO₂ incubator; then washed in PBS. The cells were fixed with 4% Paraformaldehyde in PBS for 15 minutes at room temperature and blocked with 3% BSA in PBS for 30 minutes at room temperature. Cells were stained with a Clathrin Heavy Chain Monoclonal Antibody, AlexaFluor 488 at a dilution of 5 μg/mL in blocking buffer for 1 hour at room temperature protected from light. Nuclei (blue) were stained with Hoechst Dye at a dilution of 1:10,000 in blocking buffer for 5 minutes at room temperature. Images were taken on a confocal microscope Leica TCS SP8.

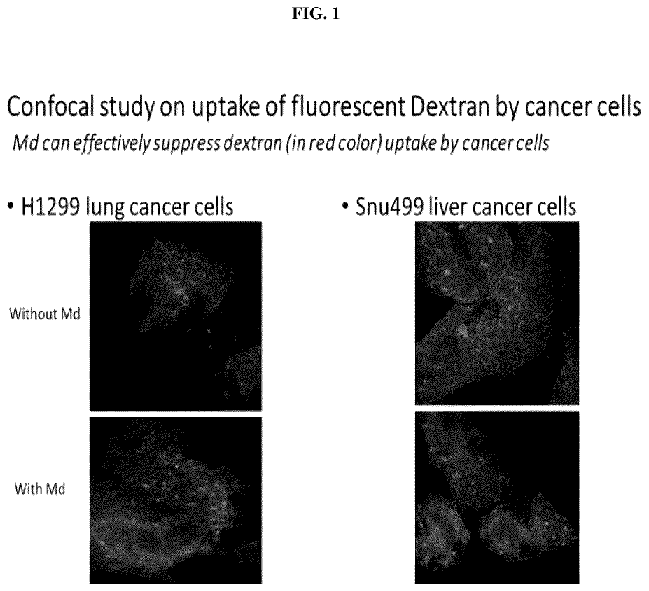

As showed in FIG. 1, the red color which represents dextran was significantly lower after adding methylenediamine dihydrochloride. The study revealed that methylenediamine dihydrochloride can effectively suppress the uptake of dextran by the two types of cancer cells (lung and liver).

### Example 2. Methylenediamine Dihydrochloride Inhibited Growth (Proliferation) of Cancer Cells

The cancer cell lines were cultured in DMEM supplemented with 10% fetal bovine serum (FBS) and 100 U/mL penicillin-streptomycin in a humidified 37° C. incubator supplemented with 5% CO₂. Cell proliferation was analyzed using MTT proliferation kit (Sigma). In brief, the cells were plated (5.0×10³ cells per well) in 96-well plates and allowed to adhere overnight. The cells were then treated with methylenediamine dihydrochloride at various concentrations (0, 1 μM, 50 μM; 100 μM. 250 μM, 500 μM and 1 mM for 24 hours before the assay. After the incubation period, 10 μL of the MTT labeling reagent (final concentration 0.5 mg/mL) was added to each well. The microplate was incubated for 4 hours in a humidified atmosphere (37° C., 5% CO₂). 100 μL of the Solubilization solution was added into each well. The plate was allowed to stand overnight in the incubator in a humidified atmosphere (37° C., 5% CO₂). Complete solubilization of the purple formazan crystals was checked and the absorbance of the samples was measured using a microplate reader with wavelength of 570 nm.

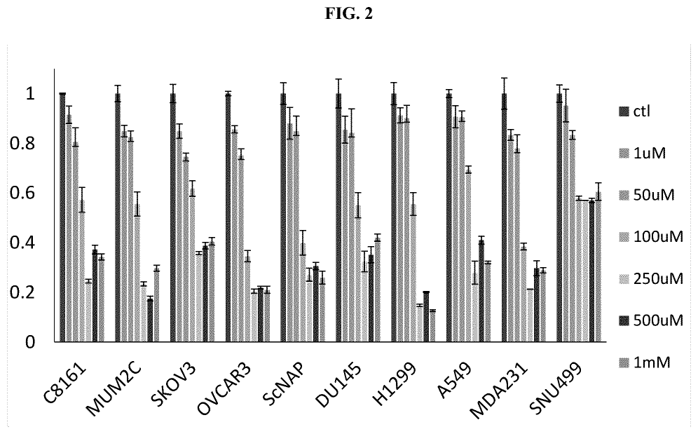

As showed in FIG. 2, methylenediamine dihydrochloride started to suppress most cancer cells' growth at the concentration of 50 μM and significantly suppressed the proliferation above 100 μM in a dose dependent pattern.

### Example 3. Methylenediamine Dihydrochloride Induced Apoptosis (Cell Death) in Melanoma and Lung Cancer

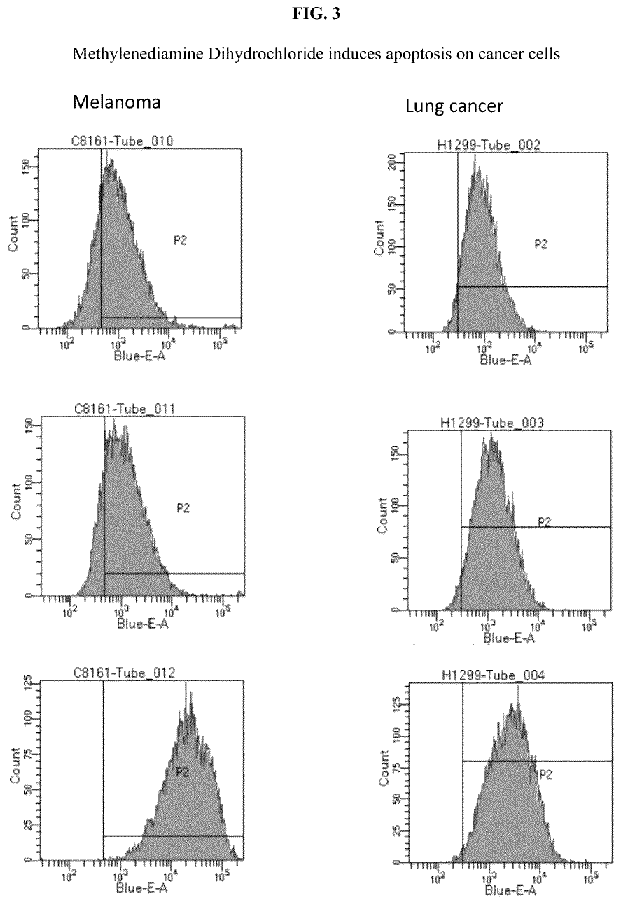

An apoptosis kit was purchased from Invitrogen lot 2208491. The study was carried out according to the company's protocol. Cells of H1299 and C8161 were cultured in DMEM/FBS medium without or with the drug at 100 μM or 500 μM and, after 24 hours, harvested and washed in cold phosphate-buffered saline (PBS). 1× annexin-binding buffer was prepared: adding 1 mL 5× annexin binding buffer to 4 mL deionized water. The washed cells were re-centrifuged. The supernatant was discarded and the cells were resuspended in 1× annexin-binding buffer. The cell density was determined and diluted in 1× annexin-binding buffer to ˜1×10⁶ cells/mL, and a sufficient volume to have 100 μL per assay was prepared. 5 μL Alexa Fluor® 488 annexin V was added to each 100 μL of cell suspension, and the cells were incubated at room temperature for 15 minutes. After the incubation period, 400 μL 1× annexin-binding buffer was added, mixed gently and kept on ice. The stained cells were analyzed with flow cytometry, and the fluorescence emission at 530 nm was measured. As showed in FIG. 3, compared with the control, methylenediamine dihydrochloride stimulated more cells with fluorescent which indicated more cells in apoptosis. More fluorescence was observed at the higher drug concentration of 500 μM than 100 μM. The results showed that methylenediamine dihydrochloride significantly induced apoptosis in these two cancer cells in a dose dependent manner.

### Example 4. Suppression of Blood Cancer Cells

50000 cells of each cell line were plated per well in a 24 well plate, treated with methylenediamine hydrochloride at different concentrations (0 as control, 50 μM, 100 μM and 500 μM) for 24 hours, then the living cells were counted. Jurkat: T cell leukemia, HL-60: acute leukemia cell; MV-411: B-myelomonocytic leukemia.

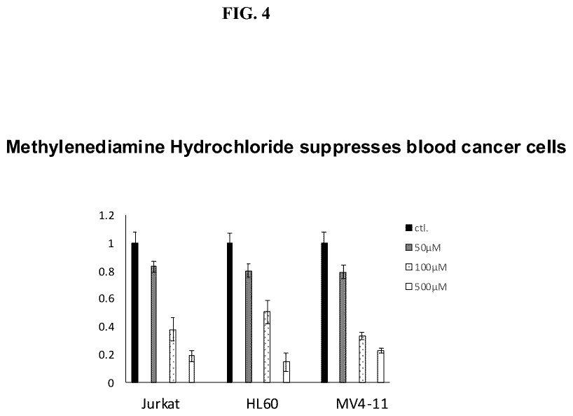

In FIG. 4, the four columns represent number of living cells at different concentrations of methylenediamine hydrochloride: from left to right, when concentration increased, fewer living cells were observed for all the three blood cancer cell lines. The suppression was more significant at higher concentrations in a dose dependent manner. This showed a significant suppression of three blood cancer cells (Jurkat, HL-60 and MV-411) by methylenediamine hydrochloride.

### Example 5. Methylenediamine Dihydrochloride Inhibited Tumor Growth in the Mouse Model with Liver Cancer

H22, a mouse liver cancer cell line from ATCC, was cultured in 1640 medium with 10% BSA at 37° C., 5% CO₂. The cells were harvest 48 hours later and diluted to 1×10⁶ cells/mL with the medium. Kunming mice, male, and body weight of 20 to 23 grams were used for the in vivo study. The animals were allowed free access to food and water throughout the study. All experimental protocols described here were approved by the Ethics Review Committee for Animal Experimentation. 0.2 mL of 1×10⁶/ml cell suspension was inoculated into the abdominal cavity of the Kunming mice. The H22 ascites was transplanted 5 days later. The H22 cells in the ascites were harvested, diluted to a concentration of 1×10⁷/mL with sterilized NS, then 0.2 mL was injected subcutaneously into the right armpit region of the mice. The transplanted tumor could be observed 5 days later. The animals were randomly divided into 3 groups with 7 mice in each and received the drug through mouth daily. Group 1: normal saline; Group 2: Methylenediamine Dihydrochloride low dose of 120 mg/Kg; Group 3: methylenediamine dihydrochloride high dose of 240 mg/Kg. Two perpendicular dimensions (length and width) of the subcutaneous transplantation tumors were measured in every mouse at day 3, day 6 and day 9 to calculate the tumor volume which was compared among groups (volume=½×length×width²). As showed in Table 1, methylenediamine dihydrochloride successfully suppressed tumor growth in the mouse liver cancer model with the suppression being dose dependent. The tumor suppression rate was calculated as: tumor volume in normal saline group minus tumor volume in methylenediamine dihydrochloride treated group, then divided by tumor volume of normal saline group. Methylenediamine dihydrochloride achieved 30.5% suppression of liver cancer in the lower dose and 36.8% in the higher dose.

TABLE 1
Methylenediamine Dihydrochloride suppressed tumor
growth of liver cancer in mice

| | | | | Tumor suppression |
|---|---|---|---|---|
| | Day 3 | Day 6 | Day 9 | rate at day 9 |
| Normal saline | 0.47 | 1.19 | 1.74 | — |
| Methylenediamine | 0.43 | 0.85 | 1.21 | 30.5% |
| Dihydrochloride | | | | |
| 120 mg/Kg | | | | |
| Methylenediamine | 0.41 | 0.78 | 1.10 | 36.8% |
| Dihydrochloride | | | | |
| 240 mg/Kg | | | | |

Average tumor volume (cm³) after injection of methylenediamine dihydrochloride

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for treating cancer, or a related disease or condition, comprising administering to a subject in need thereof a therapeutically effective amount of a pharmaceutical composition comprising methylenediamine (diaminomethane), or a pharmaceutically acceptable form thereof.

2. A method for reducing the risk of or delaying recurrence of cancer after remission or surgery, comprising administering to a subject in need thereof a therapeutically effective amount of a pharmaceutical composition comprising methylenediamine (diaminomethane), or a pharmaceutically acceptable form thereof.

3. (canceled)

4. A method for suppressing growth of cancer cells, comprising administering to a subject in need thereof a pharmaceutical composition comprising methylenediamine (diaminomethane), or a pharmaceutically acceptable form thereof.

5. The method of claim 1, wherein methylenediamine is in the form as a pharmaceutically acceptable salt.

6. The method of claim 5, wherein methylenediamine is in the form of methylenediamine dihydrochloride.

7. The method of claim 1, wherein cancer is a solid tumor.

8. The method of claim 7, wherein the solid tumor is selected from the group consisting of: lung cancer, liver cancer, skin cancer, ovarian cancer, prostate cancer and breast cancer.

9. The method of claim 1, wherein cancer is a blood cancer.

10. The method of claim 9, wherein the blood cancer is selected from the group consisting of: leukemia, lymphoma and myeloma.

11. The method of claim 1, wherein the administration is oral, subcutaneous, intramuscular, intratumoral, intravenous, or inhaled administration.

12. (canceled)

13. The method of claim 6, wherein methylenediamine dihydrochloride is administered at a daily dosage in the range of about 10 mg to about 1,000 mg for a time period of about 7 to about 180 days.

14. The method of claim 6, wherein methylenediamine dihydrochloride is administered at a daily rising dosage in the range of about 10 mg to about 1,000 mg for a time period of about 7 to about 180 days.

15. The method of claim 14, wherein methylenediamine dihydrochloride is administered at a daily rising dosage in the range of about 15 mg to about 500 mg for a time period of about 7 to about 180 days.

16. The method of claim 15, wherein methylenediamine dihydrochloride is administered at a daily rising dosage in the range of about 20 mg to about 250 mg for a time period of about 7 to about 180 days.

17. The method of claim 1, further comprising administering to the subject a second therapy or a second therapeutic agent.

18. The method of claim 1, in combination with one or more of chemotherapy, hormone therapy, radiation therapy, and immunotherapy.

19-25. (canceled)

26. A pharmaceutical composition comprising methylenediamine (diaminomethane), or a pharmaceutically acceptable form thereof, in a therapeutically effective amount and a pharmaceutically acceptable excipient, carrier, or diluent.

27. The pharmaceutical composition of claim 26, wherein methylenediamine is in the form as a pharmaceutically acceptable salt.

28. (canceled)

29. A unit dosage form comprising a pharmaceutical composition according to claim 29.

30-32. (canceled)

33. A kit comprising a unit dosage form of claim 29 and a dosage unit form of a second therapeutic agent and instructions for administration thereof.

34-39. (canceled)
